(19) United States
(12) Patent Application Publication
(10) Pub. No.: US 2023/0000693 A1
(43) Pub. Date: Jan. 5, 2023

(54) COMPOSITE WEB

(71) Applicant: GDM S.p.A., Bologna (BO) (IT)

(72) Inventors: Matteo PIANTONI, Bologna (IT); Alessandro ZAVALLONI, Bologna (IT)

(21) Appl. No.: 17/852,830

(22) Filed: Jun. 29, 2022

(30) Foreign Application Priority Data
Jun. 30, 2021 (IT) 102021000017132
Jun. 30, 2021 (IT) 102021000017159

(51) Int. Cl. A61F 13/15

(52) U.S. Cl. CPC A61F 13/15699; A61F 13/15739; B29L 2031/4878

(57) ABSTRACT

A composite web including: a first continuous web; at least one molded segment having at least one protrusion and a base portion at least partly delimiting the protrusion, the molded segment being obtained from a second continuous web; the molded segment being joined to the first continuous web by the base portion, the first continuous web and the molded segment delimiting at least one cavity at the protrusion.

This application claims priority to Italian Patent Application 102021000017159 filed Jun. 30, 2021 and Italian Patent Application 102021000017132 filed Jun. 30, 2021, the entirety of both are incorporated by reference herein.

### BACKGROUND OF THE INVENTION

This invention relates to a composite web used to make absorbent sanitary articles, an absorbent sanitary article comprising the composite web, a method for the production of and a unit for forming the composite web.

Generally speaking, absorbent sanitary articles essentially comprise a topsheet, a backsheet and an absorbent core disposed between the topsheet and the backsheet. The topsheet is usually the layer that is in contact with the wearer, while the backsheet is the outer layer of the article.

Known in the prior are absorbent articles, like the one shown, for example, in document WO2016040091A1, which comprise a topsheet comprising what is known as a three-dimensional layer, with bubble-like protrusions obtained by deforming a web using embossing technology; the bubbles allow improving the comfort of the wearer of the absorbent article.

To ensure that the sheet subjected to embossing keeps the desired shape, the topsheet usually also comprises a second layer or base layer joined to the embossed web.

Disadvantageously, the production of webs for sanitary articles including a topsheet that comprises an unbroken three-dimensional layer applied to a base layer, also unbroken, involves large amounts of material and high production costs.

There is a need to make webs for sanitary articles that are more economical while still ensuring wearer comfort.

### SUMMARY OF THE INVENTION

This invention therefore has for an aim to meet the above mentioned need.

At least the aim specified is achieved by a composite web, an article, a method for the production of and a unit for forming the composite web in accordance with the independent claims.

The dependent claims correspond to possible different embodiments of the invention.

According to an aspect, the disclosure relates to a composite web, in particular a composite web used for the production of absorbent sanitary articles such as, for example, diapers and the like.

According to an aspect, the composite web comprises a first continuous web that acts as support or matrix.

According to an aspect, the composite web comprises at least one molded segment. Preferably, the molded segment is obtained from a second continuous web.

According to an aspect, the molded segment has at least one protrusion or protuberance and a base portion which at least partly delimits the protrusion itself.

According to an aspect, the molded portion is joined to the first continuous web by the base portion.

According to an aspect, the first continuous web and the molded portion delimit at least one cavity at the protrusion.

According to an aspect, the base portion is outside the protrusion.

According to an aspect, in the composite web, in particular in a segment thereof when used in a sanitary article, the protrusions are in contact with the wearer and the first web is in contact with the absorbent core.

In other words, the protrusion protrudes from the base portion towards the wearer; this allows enhancing the comfort of the absorbent article that incorporates the composite web.

According to an aspect, the molded segment comprises a multiplicity of protrusions delimited by a multiplicity of base portions.

According to an aspect, the first continuous web and the molded portion delimit a multiplicity of cavities at the protrusions.

According to an aspect, the multiplicity of protrusions defines a motif on the molded segment.

In other words, the multiplicity of protrusions defines a pattern on the molded segment.

In an example, the protrusion may have a bubble shape.

In another example, the protrusion may have a wavy shape.

Other examples of shapes that the protrusion may have are the following: circular, diamond, oval, teardrop, elliptical, heart, triangular.

Advantageously, the protrusions improve the comfort of the wearer in contact therewith.

According to an aspect, the composite web comprises a multiplicity of molded segments joined to the first continuous web.

Advantageously, joining the first continuous web to the molded segment allows fixing the deformation (that is the molding) imparted to the molded segment at least at the protrusion.

Advantageously, using molded segments joined to the first continuous web, which keeps them in shape, allows reducing the amount of material needed to make a comfortable topsheet.

In particular, using molded segments instead of an entirely molded continuous web allows reducing the amount of material needed.

Advantageously, reducing the amount of material used allows lowering production costs.

According to an aspect, the molded segment is molded using embossing technology.

For example, a continuous web can be cut and spaced (using known slip and cut methods) to obtain segments of web which can then be molded by making the segments pass between two rollers provided with male and female portions to obtain molded segments.

For example, a continuous web can be molded to obtain an embossed web and this continuous web can then be cut and spaced (for example, using known slip and cut methods) so as to obtain molded segments of first web.

According to an aspect, the molded segment comprises non-woven fabric.

According to an aspect, the first web comprises non-woven fabric.

By “non-woven fabric” is meant a material whose fibers are randomly oriented.

The first web and/or the molded segment may comprise different materials such as, for example: carded fibers, non-woven fabrics, cellulose fibers, viscose, polyester, cotton, polyamide, and microfiber.

According to an aspect, the composite web comprises at least one weld. Advantageously, the weld allows joining together the base portion of the molded segment and the first continuous web.

For example, welding may be accomplished by thermal welding or thermomechanical welding or ultrasonic welding.

According to an aspect, the composite web comprises at least one adhesive layer disposed between the base portion of the molded segment and the first continuous web.

Advantageously, the adhesive layer allows joining together the base portion of the molded segment and the first continuous web.

According to an aspect, the description regards an absorbent article essentially comprising: a topsheet, an impermeable backsheet and an absorbent core disposed between the topsheet and the backsheet.

According to an aspect, the topsheet comprises a segment of a composite web according to one or more of the aspects set out above.

According to an aspect, the first continuous web is disposed between the molded segment and the absorbent core.

According to an aspect, an acquisition and distribution Layer (ADL) may be provided between the first continuous web and the absorbent core.

Advantageously, the presence of an ADL allows improving the movement of the liquids which pass through the topsheet to be collected in the absorbent core.

For example, the molded segment may be made in the form of what is known as a bubble topsheet, that is to say, a topsheet with bubbles on it to enhance the comfort of the wearer.

Advantageously, making an absorbent article whose topsheet is made in accordance with one or more of the above-mentioned aspects allows reducing the amount of material needed to produce the absorbent article.

Advantageously, reducing the amount of material needed to make the article allows reducing production costs.

According to an aspect, the first continuous web used for the topsheet joined to the base portion of the molded segment ensures that the segment keeps the required shape.

Advantageously, ensuring that the protrusions on the molded segment remain in shape enhances the comfort of the wearer whose skin will be in contact with the protrusions of the sanitary article.

This description relates to a method for the production of composite webs (in particular for making absorbent sanitary articles) according to one or more of the above-mentioned aspects.

According to an aspect, the method comprises a step of feeding a first continuous web.

According to an aspect, the method comprises a step of feeding at least one molded segment of a second continuous web.

According to an aspect, the molded segment has at least one protrusion which is at least partly delimited by a base portion.

According to an aspect, the method comprises a step of joining the molded segment to the first continuous web at the base portion to delimit at least one cavity between the first continuous web and the molded segment at the protrusion.

Advantageously, joining the first continuous web to the base portion of the molded segment allows ensuring that the protrusion keeps its shape.

According to an aspect, the method comprises a step of molding a segment of a second continuous web to obtain the molded segment.

Advantageously, molding the segment allows making protrusions that improve the comfort of the wearer of the end product comprising the portion of molded web.

According to an aspect, the step of molding is performed using embossing technology.

For example, molding can basically be performed using a first roller that is peripherally provided with a plurality of suction hollows, and a second roller that is peripherally provided with a plurality of teeth that are substantially shaped to match the suction hollows.

According to an aspect, the method comprises a step of feeding the second continuous web to cut and space it in such a way as to obtain segments of second continuous web.

According to an aspect, the step of cutting and the step of spacing to obtain segments of second web is performed using, for example, the slip and cut method.

According to an aspect, the step of joining the molded segment to the first continuous web comprises a step of welding the molded segment to the first continuous web at least at the base portion.

Welding may be, for example, thermal, thermomechanical or ultrasonic welding.

According to an aspect, the step of joining the molded segment to the first continuous web comprises a step of disposing at least one layer of adhesive between the molded segment and the first continuous web.

According to an aspect, the step of joining the molded segment to the first continuous web comprises a step of joining a discrete succession of molded segments to the first continuous web.

Advantageously, joining the molded segments to the first continuous web allows fixing and keeping the 3D pattern, defined by the protrusions, imparted to the segments.

Advantageously, keeping the 3D pattern molded on the molded segments ensures enhanced comfort for the wearer, whose skin will be in contact with the protrusions.

Advantageously, joining molded segments instead of a continuous molded web allows reducing the amount of material used for the production of the sanitary article.

Advantageously, using a smaller amount of material allows reducing production costs.

This description relates to a forming unit for making composite webs according to one or more of the above mentioned aspects.

According to an aspect, the forming unit comprises an applicator device configured to join a molded segment to a first continuous web.

According to an aspect, the molded segment has a pattern which comprises at least one protrusion and one base portion.

According to an aspect, the forming unit comprises a production device for making the molded segment.

According to an aspect, the production device comprises a molding device for molding the pattern on the segment and a cutting and spacing device for cutting and spacing a second continuous web to make at least one segment or a plurality of segments of second continuous web.

For example, the molding device may be disposed downstream of the cutting and spacing device in a feed direction of the second continuous web so as to mold the pattern on the segment or segments of the plurality of segments.

In an example, the molding device may be disposed upstream of the cutting and spacing device in a feed direction of the second continuous web so as to mold the pattern on the second continuous web.

The molding device is, for example, an embossing device.

According to an aspect, the molding device comprises a first drum and a second drum positioned relative to each other to define a gap through which the segment of second web or the second web can pass.

Advantageously, the molding device allows making the three-dimensional pattern, made up of the protrusions on the molded segment, thereby enhancing the comfort of the wearer, whose skin will be in contact with the molded segment of the sanitary article being worn.

According to an aspect, the segments of second web or the second web are molded by conveying them between the drums of the molding device.

According to an aspect, the axes of the drums of the molding device are parallel to each other.

According to an aspect, the first drum is rotatable about a respective axis, for example, anticlockwise, and the second drum is rotatable about a respective axis, for example, clockwise (or vice versa).

According to an aspect, the applicator device is located downstream of the production device in a feed direction of the molded segment.

According to an aspect, the applicator device is configured to join the molded segment to the first continuous web at the base portion.

According to an aspect, the cutting and spacing device comprises an anvil and a rotary knife acting in conjunction with the anvil.

According to an aspect, the cutting and spacing device, in particular the anvil, is configured to space the segments of second web.

According to an aspect, the segments are cut and spaced, for example, using a slip and cut method on the anvil itself.

According to an aspect, the spaced segments continue along their feed path and pass through the gap of the molding device so as to obtain molded segments of second web.

According to an aspect, the applicator device comprises at least one applicator drum tangent to the first drum or to the second drum.

According to an aspect, the applicator device comprises a welder for welding the molded segment to the first continuous web by thermal or thermomechanical welding.

For example, welding is ultrasonic welding.

According to an aspect, the applicator device comprises an adhesive dispenser, the adhesive dispenser being located downstream of the production device in the feed direction of the molded segment.

Advantageously, the applicator device allows keeping the shape that the molding device has imparted to the molded segment.

According to an aspect, the first drum and the second drum of the molding device are provided, respectively, with male portions and female portions, which are shaped to match each other and configured to mold the 3D pattern (defined by the protrusions) on the molded segment when the web passes through the gap.

The male and female portions are shaped according to the shape which the protrusions are intended to have.

### BRIEF DESCRIPTION OF THE DRAWINGS

The main features of the invention are more apparent from the detailed description which follows, with reference to the accompanying drawings which illustrate a preferred, non-limiting embodiment of the invention, and in which:

FIG. 1 illustrates a detail of a composite web according to the description in a schematic cross sectional view;

FIG. 2 illustrates a detail of a composite web according to the disclosure in a schematic perspective view;

FIG. 3 illustrates a detail of a composite web according to the disclosure in a schematic perspective view;

FIG. 4 illustrates an absorbent article according to the description in a schematic plan view;

FIG. 5 illustrates a unit for forming a composite web according to the description in a schematic front view;

FIG. 6 illustrates a detail of a unit for forming a composite web according to the description in a schematic view;

FIG. 7 illustrates a detail of a unit for forming a composite web according to the description in a schematic view;

FIG. 8 illustrates an absorbent article according to the description in a schematic cross sectional view.

### DESCRIPTION OF THE PREFERRED EMBODIMENTS

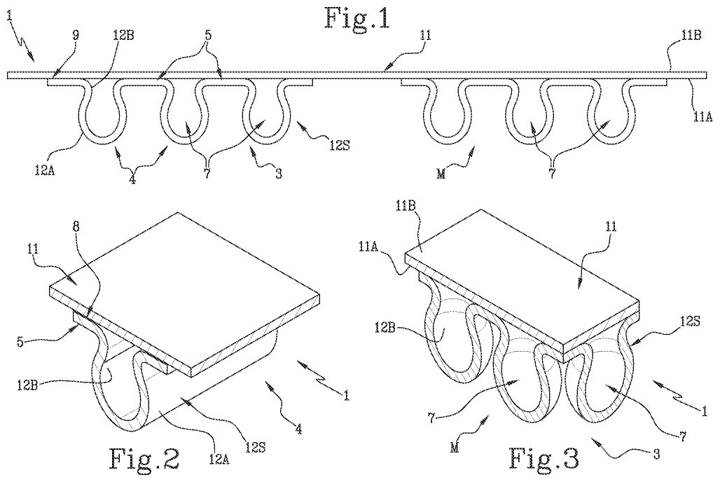

With reference to the accompanying drawings and, in particular, FIG. 1, the numeral 1 denotes a composite web according to the description.

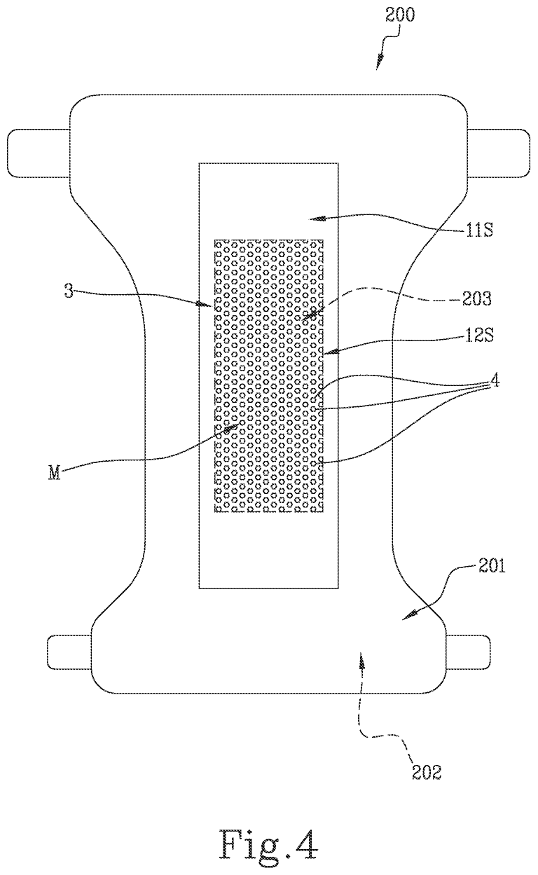

The composite web 1 is preferably intended for the production of absorbent sanitary articles such as diapers, an example of which, labelled 200, is illustrated in FIG. 4.

The composite web 1, as explained in more detail below, allows making what are known as three-dimensional or 3D topsheets for diapers.

The composite web 1 comprises a first continuous web 11 and a molded segment 12S, that is to say, a segment with a pattern M stamped on it.

The molded segment 12S is obtained from a segment 12L of a second continuous web 12. The segment 12S has a first face 12A and a second face 12B.

Basically, the molded segment 12S has at least one protrusion 4 which is at least partly delimited by a base portion 5, outside the protrusion 4.

The protrusion 4 protrudes from the first face 12A relative to the base portion 5.

With reference to the accompanying drawings, the cross section of the composite web 1 is greatly scaled up in the illustrations so as to make it easier to understand this description.

The molded segment 12S defines what is known as the 3D part of the topsheet, made to improve the comfort of the diaper, thanks to the protrusions 4.

The segment 12S thus defines a molded portion 3 for the composite web 1.

The first continuous web 11 has a first face 11A and a second face 11B.

The first face 11A is joined to the second face 12B of the segment 12S by the base portions 5.

The second face 11B is in contact preferably with the absorbent core 203 of the absorbent sanitary product 200 or with an ADL, if any.

The composite web 1 has at least one cavity 7 which is delimited by the molded segment 12S and by the web 11 at the protrusions 4.

In other words, the cavity 7 is delimited by the second face 12B of the segment 12S at the protrusion 4 and by the first face 11A of the first continuous web 11.

In an embodiment, the segment 12S, on its first face 12A, has a multiplicity of protrusions 4, delimited by a respective multiplicity of base portions 5 surrounding the protrusions 4.

In this embodiment, the composite web 1 has a multiplicity of cavities 7 delimited by the segment 12S and by the first continuous web 11 (at the multiplicity of protrusions 4).

The multiplicity of protrusions 4 defines the motif M, that is, a pattern that will constitute the three-dimensional portion of the topsheet.

The wearer, when wearing an absorbent sanitary product 200 that comprises the composite web 1, is in contact with the protrusions present on the first face 12A of the segment 12S.

Advantageously, the protrusions 4 improve the comfort for the wearer who is wearing the product and comes into contact with the protrusions.

In an embodiment, the molded segment 12S comprises non-woven fabric.

In an embodiment, the molded segment 12S comprises polymer fibers.

In an embodiment, the first continuous web 11 comprises non-woven fabric.

In an embodiment, the first continuous web 11 is a liquid acquisition and distribution layer (ADL).

The shape imparted to the segment 12S by molding is maintained by gluing the segment 12S to the first continuous web 11.

In an embodiment, the composite web 1 comprises a discrete succession of segments 12S joined to the first web 11.

The composite web 1 has a succession of molded portions 3 (at the segments 12S), kept in the required shape by the first continuous web 11.

In an absorbent article 200 that uses a segment of the web 1 as topsheet, the segment will have a molded portion 3, enhancing the comfort of the wearer, at the molded segment 12S.

In an embodiment, the protrusions 4 of the segment 12S have a bubble shape, illustrated, for example, in FIG. 3.

In an embodiment, the protrusions 4 of the segment 12S have a wavy shape, illustrated, for example, in FIG. 2.

In an embodiment, the waves molded on the segment 12S comprise waves with crests 4A that are parallel to the main direction of extension of the segment 12S.

In an embodiment, the waves molded on the segment 12S comprise waves with crests 4A that are transverse to the main direction of extension of the segment 12S.

The protrusions 4 may have different shapes: for example, diamond, heart, oval, teardrop, elliptic, shamrock and others.

The segments 12S and the first web 11 are joined, for example, by a weld 9, shown in FIG. 1, and/or by a layer of adhesive 8, illustrated in FIG. 2.

In an embodiment, the segments 12S and the web 11 are joined by welding, in particular thermal welding.

In an embodiment, the segments 12S and the web 11 are joined by ultrasonic welding.

The layer of adhesive 8 is disposed between the segment 12S and the continuous web 11, preferably between the base portions 5 that delimit the protrusions 4 and the web 11.

This description relates to an absorbent article illustrated, for example, in FIG. 4 and denoted by the reference numeral 200.

The absorbent article 200, described only insofar as necessary for understanding this invention, essentially comprises: a topsheet 201, an impermeable backsheet 202 and an absorbent core 203 disposed between the topsheet 201 and the backsheet 202.

The topsheet 201 is the part of the article 200 which, in use, is in contact with the wearer.

The topsheet 201 comprises a segment of a composite web 1 according to one or more of the features described above.

The composite web 1 is cut to make a segment of composite web to be assembled in the article 200.

The segment of composite web comprises at least one molded segment 12S and one segment 11S of the first continuous web 11.

In an embodiment, the molded segment 12S is smaller in length than the segment 11S.

In an embodiment, the molded segment 12S is smaller in width than the segment 11S.

As illustrated, for example, in FIG. 8, the segment 11S included in the composite web 1, is disposed between the molded segment 12S (also included in the composite web 1) and the absorbent core 203.

In an embodiment, not illustrated, the absorbent article 200 comprises an ADL (that is, a liquid acquisition and distribution layer) disposed between the segment 11S and the absorbent core 203. Preferably, the ADL is discrete and disposed at the molded segment 12S.

Advantageously, the presence of an ADL allows acquiring the liquids and directing them towards an absorbent core, thereby increasing the efficiency of the absorbent sanitary product 200.

This description relates to a method for the production of a composite web 1. The composite web 1 is preferably used for making absorbent sanitary articles such as, for example, the article 200.

The method essentially comprises the following steps:
feeding a first continuous web 11,
feeding at least one molded segment 12S of a second continuous web 12, where the segment 12S has at least one protrusion 4 delimited at least partly by a base portion 5,
joining the segment 12S to the first web 11 at the base portion 5 to delimit at least one cavity 7 between the web 11 and the segment 12S at the protrusions 4.

In an embodiment, the method comprises a step of molding a segment 12L of second continuous web 12 to obtain the molded segment 12S.

Preferably, the method, in particular the step of molding, comprises a step of embossing the segment 12L, that is, stamping or molding a 3D pattern on it.

The method used to mold the segment is commonly known as embossing.

For example, the step of molding can basically be performed using a first roller that is peripherally provided with a plurality of suction hollows, and a second roller that is peripherally provided with a plurality of teeth that are substantially shaped to match the suction hollows.

In an embodiment, the method comprises the step of feeding the second continuous web 12.

In an embodiment the method comprises a step of cutting and spacing the second continuous web 12 to obtain segments 12L of second web 12.

The segments 12L are subsequently molded to obtain segments 12S provided with protrusions 4 delimited by base portions 5.

The step of cutting and spacing can be carried out, for example, using a method known as slip and cut, that is to say, a method of cutting and sliding whereby (discrete) segments are cut from the web and then spaced apart.

In an embodiment, the step of joining at least one molded segment 12S to the first continuous web 11 comprises a step of welding the segments 12S to the web 11 at the base portion 5.

In an embodiment, the step of welding the segments 12S to the web 11 is carried out by ultrasonic welding or thermomechanical welding.

In an embodiment, the step of joining the segments 12S to the continuous web 11 comprises a step of disposing a layer of adhesive 8 between the segments 12S and the web 11.

Joining the segments 12S to the web 11 allows fixing and keeping the 3D pattern imparted to the segment 12S by molding.

In an embodiment, a succession of suitably sized and spaced segments 12S is applied to the web 11.

In an embodiment, the step of joining the segment 12S to the web 11 comprises a step of joining a discrete succession of segments 12S to the first continuous web 11.

The first continuous web 11 is joined at the base portions 5 of the molded segment 12S, in particular, it is joined on the side opposite the protrusions 4, defining the molded portion 3 for the composite web 1.

In an embodiment, the method comprises the steps of molding the continuous web 12 and cutting and spacing the continuous web 12 to obtain segments 12S.

This description relates to a forming unit 100 for making composite webs, for example of the type like the composite web 1.

The unit 100 essentially comprises a production device 101 and an applicator device 110.

The applicator device 110 is configured to join a molded segment 12S, provided with a pattern M that comprises at least one protrusion 4 and one base portion 5, to a first continuous web 11.

The production device 101 for making the segment 12S comprises a molding device 102 for molding the pattern M and a cutting and spacing device 103 configured to cut and space a second continuous web 12 to make at least one segment or a plurality of segments 12L of the second continuous web 12.

The device 102 is of a type known in the trade and is also called embossing device.

In an embodiment, the device 102 is disposed downstream of the device 103 in a feed direction V12 of the second web 12 so as to mold the pattern M on the segment 12L.

In an embodiment not illustrated, the device 102 is disposed upstream of the device 103 in the feed direction V12 to mold the pattern M on the web 12. That way, the segments 12S that are cut and spaced are already molded.

The device 102 comprises a first drum 104 and a second drum 105 positioned relative to each other to define a through-gap 106 for passage of the segment 12L or of the web 12.

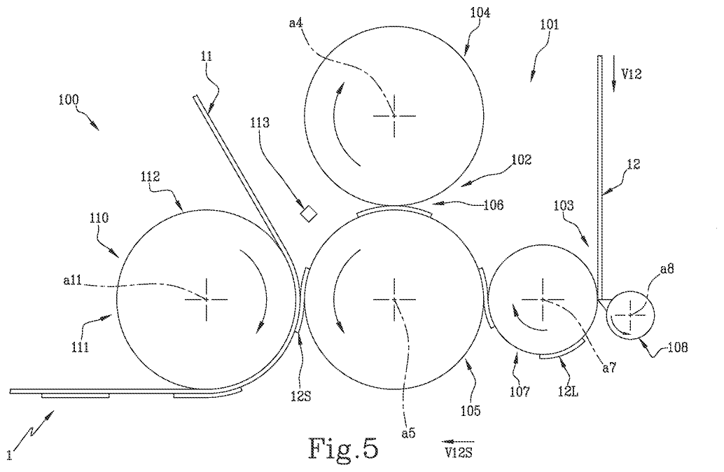

In the example illustrated in FIG. 5, the first drum 104 is rotatable clockwise about an axis a4, while the second drum 105 is rotatable anticlockwise about an axis a5, parallel to a4. For simplicity, the drums 104 and 105 are considered to be substantially tangent.

In an embodiment, the first drum 104 and the second drum 105 are provided, respectively, with the male portions and the female portions, which are shaped to match each other and to mold the pattern M (formed of the protrusions 4).

The male-female portions present on the drums 104 and 105 allow making the protrusions 4 on the segment 12S.

The male and female portions are shaped according to the shape which the protrusion 4 is intended to have.

In an embodiment, the segment 12S is thus molded by conveying it between the drums 104, 105.

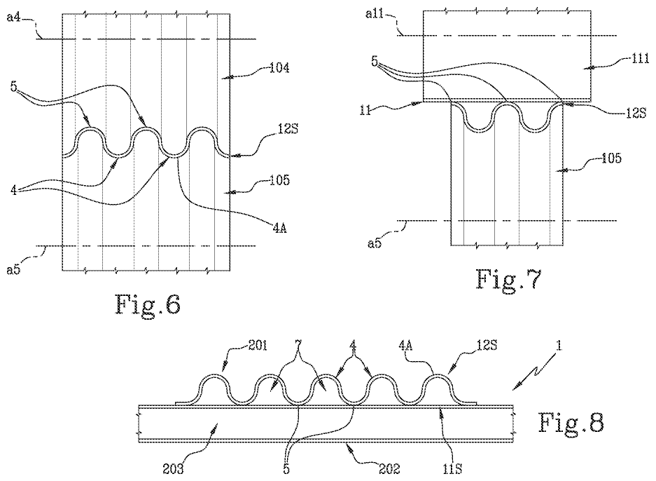

The drums 104, 105 shown in FIG. 6, for example, are shaped to make wavy protrusions 4.

In an embodiment, the drum 104 is patterned and the drum 105 acts as anvil.

The segment 12L advances and passes through the gap 106 so that the outside surfaces of the drums 104, 105 act in conjunction to engage the segment 12L to impart the 3D pattern to it.

The device 110 is configured to join the segment 12S to the web 11 at the base portion 5.

In an embodiment, the device 110 is located downstream of the production device 101 in a feed direction V12S of the molded segment.

In an embodiment, as illustrated in the example of FIG. 5, the device 103 comprises an anvil 107, preferably in the form of a drum that is rotatable clockwise about an axis a7, and a rotary knife 108 that is rotatable anticlockwise about an axis a8, and acts in conjunction with the anvil 107.

The knife 108 cuts the continuous web 12 to form the segments which are suitably spaced, for example on the anvil 107 itself with a known slip and cut method not further described.

In an embodiment, the device 103, in particular the anvil 107, is configured to space the segments 12L.

The device 110 comprises at least one applicator drum 111 that is tangent to the first drum 104 or to the second drum 105.

The applicator drum 111 is rotatable about an axis all clockwise in the example illustrated in FIG. 5 and is located downstream of the drum 105 in the feed direction V12S.

In an embodiment, the drum 111 is tangent to the drum 105 and allows carrying out the step of joining the segments 12S to the web 11, that is to say, joining the web 11 to the base portions 5 of the segments 12S, as illustrated schematically in FIG. 7.

In an embodiment, the device 110 comprises a welder 112 for welding the molded segment 12S to the first continuous web 11.

In an embodiment, the drum 112 and the drum 105 are configured to weld the segments 12S to the first web 11.

In an embodiment, the device 110 comprises an adhesive dispenser 113 located downstream of the production device 101 in a feed direction V12S of the molded segment. The dispenser 113 dispenses at least one layer of adhesive 8 so that the segments 12S can be joined to the continuous web 11 by the adhesive. For example, the dispenser 113 dispenses the adhesive to the web 11 and/or to the segments 12S.

1. A composite web comprising:
a first continuous web,
at least one molded segment having at least one protrusion and a base portion at least partly delimiting the protrusion, the molded segment being obtained from a second continuous web; the molded segment being joined to the first continuous web by the base portion, the first continuous web and the molded segment delimiting at least one cavity at the protrusion.

2. The composite web according to claim 1, wherein the molded segment comprises a multiplicity of protrusions, the protrusions being delimited by a multiplicity of base portions, the molded segment being joined to the first continuous web by the base portions, the first continuous web and the molded segment delimiting a multiplicity of cavities at the protrusions.

3. The composite web according to claim 2, wherein the multiplicity of protrusions defines a pattern on the molded segment.

4. The composite web according to claim 1, comprising a multiplicity of molded segments, the molded segments being joined to the first continuous web.

5. The composite web according to claim 1, wherein the molded segment comprises a protrusion having a bubble shape.

6. The composite web according to claim 1, wherein the molded segment comprises a protrusion having a wave shape.

7. The composite web according to claim 1, comprising at least one weld, preferably a thermal weld or a thermomechanical weld or an ultrasonic weld, the base portion and the first continuous web being joined by the weld.

8. The composite web according to claim 1, comprising at least one adhesive layer, the adhesive layer being disposed between the base portion of the molded segment and the first continuous web.

9. The composite according to claim 1, wherein the base portion is outside the protrusion.

10. An absorbent article comprising:
a topsheet,
an impermeable backsheet,
an absorbent core disposed between the topsheet and the backsheet, wherein the topsheet comprises a segment of a composite web according to claim 1, the first continuous web being disposed between the molded segment and the absorbent core.

11. A method for the production of a composite web, specifically for making absorbent sanitary articles, comprising the following steps:
feeding a first continuous web,
feeding at least one molded segment of a second continuous web, the molded segment having at least one protrusion) delimited at least partly by a base portion,
joining the molded segment to the first continuous web at the base portion to delimit at least one cavity between the first continuous web and the molded segment at the protrusion.

12. The production method according to claim 11, comprising a step of molding a segment of the second continuous web to obtain the molded segment.

13. The production method according to claim 12, further comprising the following steps:
feeding the second continuous web,
cutting and spacing the second continuous web to obtain the segment of the second continuous web.

14. The production method according to claim 11, wherein the step of joining the molded segment to the first continuous web comprises a step of welding the molded segment to the first continuous web at least at the base portion.

15. The production method according to claim 11, wherein the step of joining the molded segment to the first continuous web comprises a step of disposing a layer of adhesive between the molded segment and the first continuous web.

16. The production method according to claim 11, wherein the step of joining the molded segment to the first continuous web comprises a step of joining a discrete succession of molded segments to the first continuous web.

17. A forming unit for making a composite web, the forming unit comprising:
an applicator device configured to join a molded segment, provided with a pattern that comprises at least one protrusion, and a base portion to a first continuous web;
a production device for making the molded segment, the production device comprising a molding device for molding the pattern and a cutting and spacing device configured to cut and space a second continuous web to make at least one segment or a plurality of segments of the second continuous web, wherein the molding device is disposed downstream of the cutting and spacing device in a feed direction of the second continuous web in order to mold the pattern on the segment or wherein the molding device is disposed upstream of the cutting and spacing device in a feed direction of the second continuous web in order to mold the pattern on the second continuous web,
the molding device comprising a first drum and a second drum positioned relative to each other to define a through-gap for passage of the segment of second web or of the second continuous web;
the applicator device being configured to join the molded segment to the first continuous web at the base portion, the applicator device being disposed downstream of the production device in a feed direction of the molded segment.

18. The forming unit according to claim 17, wherein the cutting and spacing device comprises an anvil and a rotary knife acting in conjunction with the anvil.

19. The forming unit according to claim 17, wherein the cutting and spacing device, specifically the anvil, is configured to space the segments of second web.

20. The forming unit according to claim 17, wherein the applicator device comprises at least one applicator drum that is tangent to the first drum or to the second drum.

21. The forming unit according to claim 17, wherein the applicator device comprises a welder for welding the molded segment to the first continuous web.

22. The forming unit according to claim 17, wherein the applicator device comprises an adhesive dispenser, the adhesive dispenser being disposed downstream of the production device in the feed direction of the molded segment.

23. The forming unit according to claim 17, wherein the first drum and the second drum comprise male and female portions, respectively, the male and female portions being shaped to match each other so as to mold the pattern on the molded segment.
